(19) United States
(12) Patent Application Publication
(10) Pub. No.: US 2007/0243252 A1
(43) Pub. Date: Oct. 18, 2007

(54) Oral Dosage Formulations and Methods of Preparing the Same

(75) Inventor: Grant Wayne Heinicke, Rockaway, NJ (US)

Correspondence Address: CANTOR COLBURN, LLP, 55 GRIFFIN ROAD SOUTH, BLOOMFIELD, CT, 06002, US

(73) Assignee: ACTAVIS GROUP PTC HF, Hafnarfjordur (IS)

(21) Appl. No.: 11/736,048

(22) Filed: Apr. 17, 2007

Related U.S. Application Data
(60) Provisional application No. 60/792,442, filed on Apr. 17, 2006.

(51) Int. Cl. A61K 9/62

(52) U.S. Cl. 424/468

(57) ABSTRACT

Disclosed herein are methods and compositions suitable for providing zero-order release of active agents. Disclosed herein is a multiparticulate oral dosage form comprising a plurality of pulsed-release pellets, wherein the dosage form releases the active agent at a substantially constant rate following a lag time. The dosage form comprises a combination ensemble of pellets produced by combining 2 to 8 individual ensembles of pulsed-release pellets having a particular T50 and dissolution profile.

### CROSS-REFERENCE TO RELATED APPLICATIONS

This application claims the benefits of U.S. Provisional Patent Application Ser. No. 60/792,442 filed Apr. 17, 2006, which is fully incorporated herein by reference.

### BACKGROUND

Controlled-release delivery systems are well-known in the art. Many controlled release systems release the active agent at a rate that is not zero-order, that is, the rate of release changes over time. In typical non-zero-order release, the rate of release gradually decreases as the time from administration increases, providing a level of circulating active agent in the body that is not constant. Attempts have been made to provide sustained release compositions with substantially zero-order release characteristics, such as osmotic pump devices. “Osmotic pump” controlled-release formulations may be formulated using OROS (Alza Corp., Mountain View, Calif.) technology. This technology uses osmotic pressure to deliver the active agent at a controlled rate. OROS dosage formulations include a semi-permeable membrane surrounding a core that contains at least two components, one component comprising the active agent, the other comprising an osmotic push layer, such as an osmotically active polymer. Some time after the dosage form is swallowed, water enters the membrane causing the push layer to swell, releasing the active agent at a controlled rate through a hole in the membrane. Disadvantages to osmotic pump dosage forms include complex manufacture and the use of harsh solvents in their preparation.

Verapamil is an ionic calcium influx inhibitor more commonly known as a calcium channel blocking agent. The principal pharmacologic and physiologic action of verapamil is to inhibit the transmembrane influx of extracellular calcium ions across the membrane of myocardial cells and vascular smooth muscle cells. By inhibiting calcium influx, verapamil inhibits the contractile processes of cardiac and vascular smooth muscles, thereby dilating the main coronary and systemic arteries. The drug is indicated and approved by the Food and Drug Administration for the management of unstable or chronic stable angina pectoris, for the treatment of supraventricular tachyarrhythmias, and for the temporary control of rapid ventricular rate in arterial flutter or atril fibrillation. For cardiac drugs such as verapamil, a constant rate of release is desirable.

There remains a need for improved dosage forms having zero order kinetics.

### SUMMARY

A method of providing a zero-order release dosage form for an active agent, comprises combining n^{th} amounts of n ensembles of pulsed-release pellets, each of the n ensembles having a n^{th} dissolution profile with an n^{th} T50, to produce a combination ensemble of pellets that releases the active agent at a substantially constant rate following a lag time, wherein n is 2 to 8. The n^{th} ensemble of pellets comprises an n^{th} core having disposed thereon an n^{th} core composition layer, the n^{th} core composition layer comprising the active agent, and a n^{th} pulsed-release coating disposed on the n^{th} core composition layer. The lag time of release from the combination ensemble is 30 minutes to 8 hours. At least two of the n ensembles of pulsed-release pellets have n^{th} T50s that differ by at least 4 hours.

A method of increasing patient compliance comprises providing a zero-order active agent dosage form to a human patient in need thereof, wherein the dosage form is produced by a method comprising combining n amounts of n ensembles of pulsed-release pellets, each of the n ensembles having a n^{th} dissolution profile with an n^{th} T50, to produce a combination ensemble of pellets that releases the active agent at a substantially constant rate following a lag time, wherein n is 2 to 8. The n^{th} ensemble of pellets comprises an n^{th} core having disposed thereon an n^{th} core composition layer, the n^{th} core composition layer comprising the active agent, and a n^{th} pulsed-release coating disposed on the n h core composition layer. The lag time of release from the combination ensemble is 30 minutes to 8 hours. At least two of the n ensembles of pulsed-release pellets have n^{th} T50s that differ by at least 4 hours.

An active agent dosage form comprises a combination ensemble of pellets, the combination ensemble comprising n amounts of n ensembles of pulsed-release pellets, each of the n ensembles having a n^{th} dissolution profile with an n^{th} T50, wherein the combination ensemble releases the active agent at a substantially constant rate following a lag time, wherein n is 2 to 8. The n^{th} ensemble of pellets comprises an n^{th} core having disposed thereon an n^{th} core composition layer, the n^{th} core composition layer comprising the active agent, and a n^{th} pulsed-release coating disposed on the n^{th} core composition layer. The lag time of release from the combination ensemble is 30 minutes to 8 hours. At least two of the n ensembles of pulsed-release pellets have n^{th} T50s that differ by at least 4 hours.

The above-described and other features will be appreciated and understood by those skilled in the art from the following detailed description and appended claims.

### BRIEF DESCRIPTION OF THE DRAWINGS

FIG. 1 shows an idealized pulsed-release profile.

FIG. 2 shows the release of individual pellets taken from an ensemble of pellets at a given coat weight.

FIG. 3 is a dissolution profile in pH 6.8 phosphate buffer of one embodiment of diltiazem pellets.

FIGS. 4-9 are dissolution profiles of blends of diltiazem pellets.

### DETAILED DESCRIPTION

Disclosed herein are multiparticulate dosage forms having a release profile with a lag time followed by substantially zero-order release of the active agent. Once release begins, the active agent is released from the dosage form at a substantially constant rate until the active agent is substantially dissipated. In a zero-order release, the duration of the release of active agent is independent (or substantially independent) of the concentration of active agent in the delivery system. “Substantially zero-order” means within 20% of zero-order, and preferably within 10% of zero-order, for at least about six continuous hours while the active agent is being delivered, specifically for at least about eight continuous hours, more specifically for at least about ten continuous hours, and most specifically for at least about twelve continuous hours.

An active agent is a species that, when administered to a patient, confers, directly or indirectly, a physiological effect on the patient. An indirect physiological effect, for example, includes the effect of a metabolite of the active agent. Active agent includes solvates (including hydrates) of the free compound or salt, crystalline and non-crystalline forms, as well as various polymorphs. For example, an active agent can include all optical isomers and all pharmaceutically acceptable salts thereof either alone or in combination. “Pharmaceutically acceptable salts” includes derivatives of the active agent, wherein the active agent is modified by malting non-toxic acid or base addition salts thereof, and further refers to pharmaceutically acceptable solvates, including hydrates, of such compounds and such salts.

By “oral dosage form” is meant to include a unit dosage form prescribed or intended for oral administration. An oral dosage form comprises a plurality of subunits such as, for example, pellets, packaged for administration in a single dose such as a tablet, capsule, or sachet, for example. The oral dosage form optionally comprises a loading dose of the active agent in the form of, for example, a coating. A loading dose is an immediate release portion of the dosage form.

Dissolution profile as used herein, means a plot of the cumulative amount of active agent released as a function of time. The dissolution profile can be measured utilizing the Drug Release Test <724>, which incorporates standard test USP 26 (Test <711>). A profile is characterized by the test conditions selected. Thus the dissolution profile can be generated at a preselected apparatus type, shaft speed, temperature, volume, and pH of the dissolution media.

A first dissolution profile can be measured at a pH level approximating that of the stomach. A second dissolution profile can be measured at a pH level approximating that of one point in the intestine or several pH levels approximating multiple points in the intestine.

A highly acidic pH simulates the stomach and a less acidic to basic pH simulates the intestine. By the term “highly acidic pH” is meant a pH of about 1 to about 4. By the term “less acidic to basic pH” is meant a pH of greater than about 4 to about 7.5, preferably about 6 to about 7.5. A pH of about 1.2 can be used to simulate the pH of the stomach. A pH of about 6 to about 7.5, specifically about 6.8, can be used to simulate the pH of the intestine.

Release forms may also be characterized by their pharmacokinetic parameters. “Pharmacokinetic parameters” are parameters, which describe the in vivo characteristics of the active agent over time, including for example the in vivo dissolution characteristics and plasma concentration of the active agent. By “Cₘₐₓ” is meant the measured concentration of the active agent in the plasma at the point of maximum concentration. The term “Tₘₐₓ” means the time at which the concentration of the active agent in the plasma is the highest. “AUC” means the area under the curve of a graph of the concentration of the active agent (typically plasma concentration) vs. time, measured from one time to another.

By “releasable form” is meant to include immediate-release, controlled-release, and sustained-release forms. Certain release forms can be characterized by their dissolution profile. By “immediate-release”, it is meant a conventional or non-modified release in which greater than or equal to about 70% of the active agent is released within 1 hour, specifically within 30 minutes of the initiation of dissolution. By “controlled-release” it is meant a dosage form in which the release of the active agent is controlled or modified over a period of time. “Sustained-release” or “extended-release” include the release of the active agent for an extended period of time so that the dosage frequency can be reduced.

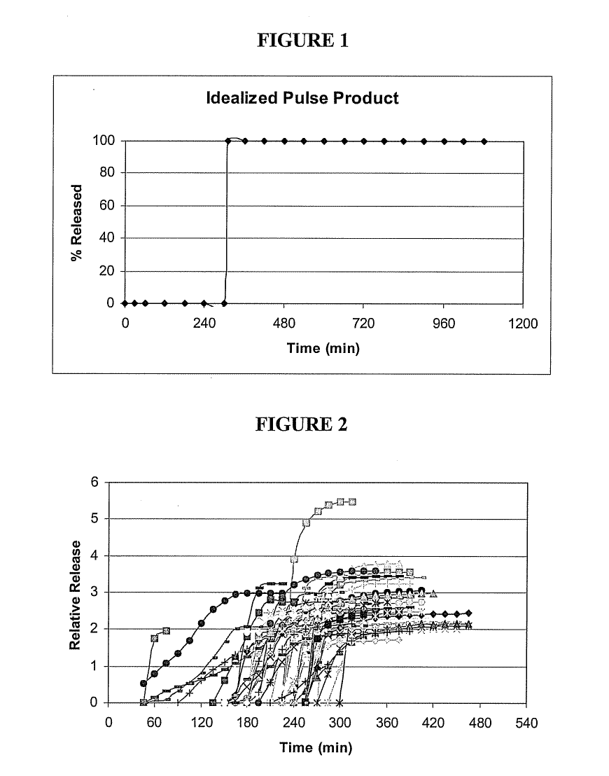

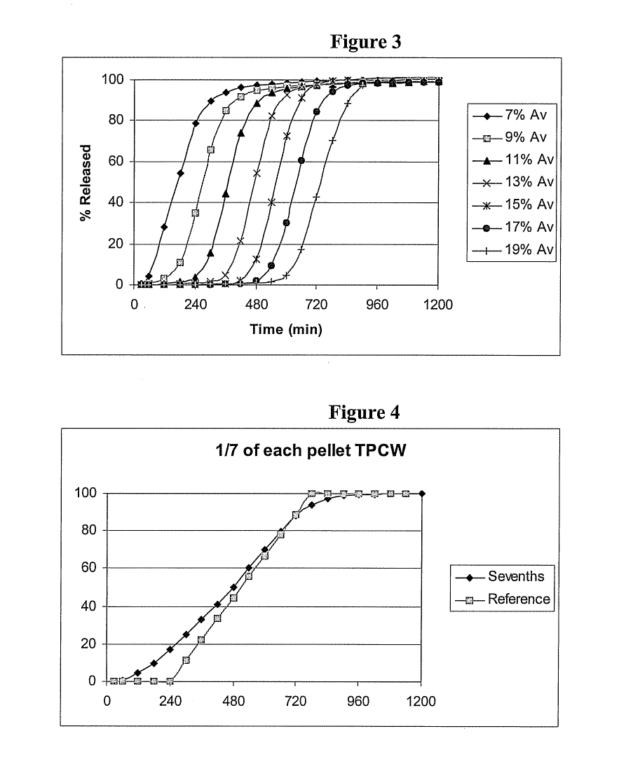

As used herein, an ensemble of “pulsed-release” pellets comprises a plurality of individual pulsed-release pellets, wherein at least about 60% of the individual pulsed-release pellets in the ensemble exhibits a lag time of substantially no release of active agent followed by immediate release of the active agent. As used herein, an ensemble of pulsed-release pellets is a population of pulsed-release pellets having substantially the same composition, e.g., a population of pellets coated so as to have the same coating weight. As shown in FIG. 1, an ideal pulsed-release active agent delivery ensemble should release the active agent completely and rapidly after a lag time. The ensemble also has a lag time of essentially no release followed by release of at least about 50% the active agent, where the release results in a dissolution profile of a given slope. However, because the ensemble is comprised of a distribution of individual pellets, release after the lag time may occur over a period of time that is somewhat longer than is typical for immediate release, or for the individual pellets themselves. However, the release of the ensemble may not be the desired extended, sustained, or controlled-release due to the pulsed-release nature of the formulation. The T50 of an ensemble of pellets is the time for the ensemble to release 50% of the active agent, measured as the halfway point between the time axis and the extent of release. In contrast to an idealized pulsed-released profile, which should be square at the top of the curve, the measured pulsed-release profiles exhibit a rounding-off at the top of the dissolution curve.

Measurement of active agent release from single pellets has yielded the dissolution profiles shown in FIG. 2. As shown in FIG. 2, once release starts, most (e.g., greater than 60%) of the pellets have a very rapid release. Without being held to theory, it is believed that the rapid release is caused by bursting of the coating to relieve pressure build up as water soaks into the center of the pellet. At some pressure, the coat bursts and the contents are released substantially immediately. What FIG. 2 illustrates is that the variability in time to release between single pulsed-release pellets can be significant, however, most pellets are immediate release after a lag time. For example, in a single ensemble of pellets having a coating weight of 11 wt %, release times of 2.5 to 5 hours were determined for the majority of the individual pellets within the ensemble.

It has been unexpectedly discovered by the inventors herein that the range of pulses from individual pulsed-release pellets is particularly suitable to manipulating active agent release profiles of dosage forms. Blending a plurality of ensembles of pulsed-release pellets with different dissolution profiles broadens the distribution of individuals, the sum of which distribution then has a unique slope, or rate of release compared to the individual ensembles. Thus, the resulting combination ensemble has a unique slope compared to its component ensembles. In a preferred embodiment, the combination ensemble has substantially zero-order release. Blending of a plurality of ensembles of pellets does not change the behavior of any individual pellet, but instead provides a combination ensemble with a release profile that cannot be achieved by one population of pellets alone. The combination ensemble comprises a sufficient number of individual ensembles blended in a manner suitable to provide a combination ensemble having substantially zero order release. In one embodiment, the combination ensemble comprises at least two individual ensembles of pulsed-release pellets, wherein each individual ensemble comprises a different coating weight. In another embodiment, the combination ensemble comprises three or more individual ensembles of pulsed-release pellets, wherein each ensemble comprises a different coating weight. In yet another embodiment, the combination ensemble comprises, 4, 5, 6, 7, 8 or more individual ensembles of pellets. Combination ensembles of pulsed-release pellets with different coating weights result in ensemble release of a wider distribution of individuals, which in turn alters the rate of release from the combination ensemble to a value not obtainable from a single coat weight itself. Each individual ensemble in a combination ensemble comprises, for example, a different coating weight of the same coating composition, a different coating composition, or a combination thereof.

In one embodiment, a zero-order release dosage form for an active agent comprises a combination ensemble of pellets, the combination ensemble comprising n amounts of n ensembles of pulsed-release pellets, each of the n ensembles having a n^{th} dissolution profile with an n^{th} T50, wherein the combination ensemble releases the active agent at a substantially constant rate following a lag time, wherein n is 2 to 8. The n^{th} ensemble of pellets comprises an n^{th} core having disposed thereon an n^{th} core composition layer, the n^{th} core composition layer comprising the active agent, and a n^{th} pulsed-release coating disposed on the n^{th} core composition layer. The lag time of release from the combination ensemble is 30 minutes to 8 hours. At least two of the n ensembles of pulsed-release pellets have n^{th} T50s that differ by at least 4 hours.

By varying the number of ensembles of pulsed-release pellets (n) as well as the properties of the n ensembles of pellets, a zero-order release profile can be achieved with 2 to 8 ensembles of pulsed release pellets, specifically 3 to 8 ensembles of pulsed-release pellets, and more specifically 3 to 6 ensembles of pulsed-release pellets. Specifically, the zero-order release is achieved in a combination ensemble of pellets wherein at least two of the n ensembles of pulsed-release pellets have n^{th} T50s that differ by at least 4 hours. With regard to the lag time, the lag time of release of the combination ensemble is determined by the earliest releasing ensemble in the combination ensemble. Thus, a first ensemble of pellets has a lag time of 30 minutes to 8 hours to give a combination ensemble having a lag time of 30 minutes to 8 hours.

A method of providing a zero-order release dosage form for an active agent, comprises combining n amounts of n ensembles of pulsed-release pellets, each of the n ensembles having a n^{th} dissolution profile with an n^{th} T50, to produce a combination ensemble of pellets that releases the active agent at a substantially constant rate following a lag time, wherein n is 2 to 8. The n^{th} ensemble of pellets comprises an n^{th} core having disposed thereon an n^{th} core composition layer, the n^{th} core composition layer comprising the active agent, and a n^{th} pulsed-release coating disposed on the n^{th} core composition layer. The lag time of release from the combination ensemble is 30 minutes to 8 hours. At least two of the n ensembles of pulsed-release pellets have n^{th} T50s that differ by at least 4 hours.

The amount of each of the n ensembles of pulsed-release pellets to give the desired constant rate of release from the combination ensemble can be determined empirically from the dissolution profiles of the individual ensembles. In one embodiment, equal amount of the n individual ensembles are employed.

The lag time for active agent release from the dosage form comprises, for example, 30 minutes to 8 hours. An 8 hour delay would be particularly beneficial for the production of chronotherapeutic dosage forms. Chronotherapeutic dosage forms are designed to be taken at bedtime such that the highest dose of the active agent occurs early in the morning, from 6 AM to noon, for example.

In one embodiment, a method of providing a zero-order release dosage form for an active agent comprises combining a first amount of a first ensemble of pulsed-release pellets having a first dissolution profile with a first T50 and a second amount of a second ensemble of pulsed-release pellets having a second dissolution profile with a second T50 to produce a combination ensemble of pellets that releases the active agent at a substantially constant rate following a lag time. The first ensemble of pellets comprises a first core having disposed thereon a first core composition layer, the first core composition layer comprising the active agent, and a first pulsed-release coating disposed on the first core composition layer. The second ensemble of pellets comprises a second core having disposed thereon a second core composition layer, the second core composition layer comprising the active agent, and a second pulsed-release coating disposed on the second core composition layer.

The dosage form optionally comprises third, fourth, fifth, sixth, seventh and eighth ensembles of pulsed-release pellets comprising a third, fourth, fifth, sixth, seventh and eighth average pulsed-release coating weight and having a fourth, fifth, sixth, seventh and eighth dissolution profile with a fourth, fifth, sixth, seventh and eighth T50.

In one embodiment, at least two of the ensembles of pulsed-release pellets have n^{th} T50s that differ by at least 4 hours. The maximum difference in T50 between the at least two ensembles is approximately the maximum time for GI transit, that is, about 24 hours. Beyond 24 hours, the pulsed-release pellets will generally not be present in the GI tract of a human subject.

In another embodiment, a first ensemble of pulsed-release pellets comprises a first average coating weight, and a second ensemble of pulsed-release pellets comprises a second average coating weight, wherein the first average coating weight and the second average coating weight differ by 1 wt % or greater, and wherein coating weights are based on the total weight of the coated pellets in each ensemble. In one embodiment, each individual ensemble of pulsed-release pellets in the combination ensemble differs in coating weight from every other individual ensemble by 1% or greater. In other embodiments, the coating weights of the plurality of ensembles of pulsed-release pellets differ by 2 wt %, 3 wt %, 4 wt %, 5 wt %, 6 wt %, 7 wt %, 8 wt %, and 9 wt %, based on the weight of the coating material. In this embodiment, the difference in coating weights results in different releases for the ensembles of pellets. In one embodiment, when the dosage form is a chronotherapeutic dosage form, the first coating weight comprises greater than or equal to 19 wt % based on the total weight of the coated pellets, and the subsequent coating weights differ from the first coating weight by 1 wt % or more. A chronotherapeutic dosage form comprises 3-6 coating weights of pellets.

In one embodiment, the method further comprises, prior to combining the two ensembles of pellets, determining the release of at least a portion of the individual pellets in the first ensemble of pulsed-release pellets, determining the release of at least a portion of the individual pellets in the second ensemble of pulsed-release pellets, and determining from the release of individual pellets in the first and second ensembles the first amount of the first ensemble of pulsed-release pellets and the second amount of the second ensemble of pulsed-release pellets to give the optimized in vitro dissolution profile. By determining the breadth of the distribution of release of individual pellets in two ensembles of pellets, one can determine a suitable ratio of the two ensembles to give a blend having a constant rate of release.

A method of increasing patient compliance comprises providing a dosage form to a human patient in need thereof, wherein the dosage form is produced by the above-described method.

An active agent dosage form comprises a combination ensemble of pellets, the combination ensemble comprising n amounts of n ensembles of pulsed-release pellets, each of the n ensembles having a n^{th} dissolution profile with an n^{th} T50, wherein the combination ensemble releases the active agent at a substantially constant rate following a lag time, wherein n is 2 to 8. The n^{th} ensemble of pellets comprises an n^{th} core having disposed thereon an n^{th} core composition layer, the n^{th} core composition layer comprising the active agent, and a n^{th} pulsed-release coating disposed on the n^{th} core composition layer. The lag time of release from the combination ensemble is 30 minutes to 8 hours. At least two of the n ensembles of pulsed-release pellets have n^{th} T50s that differ by at least 4 hours.

In one embodiment, a dosage form comprises a first amount of a first ensemble of pulsed-release pellets comprising a first average pulsed-release coating weight and having a first dissolution profile with a first T50, and a second amount of a second ensemble of pulsed-release pellets comprising a second average pulsed-release coating weight and having a second dissolution profile with a second T50, wherein the first ensemble and the second ensemble are combined to form a combination ensemble of pellets that releases the active agent at a substantially constant rate following a lag time. The first average pulsed-release coating weight and the second average pulsed-release coating weight differ by greater than 1 wt %, wherein coating weights are based on the total weight of the coated pellets. In this embodiment, the first ensemble of pellets and the second ensemble of pellets comprise a core having disposed thereon a core composition layer, the core composition layer comprising the active agent, and the pulsed-release coating disposed on the core composition layer. The lag time for active agent release from the dosage form comprises, for example, 30 minutes to 8 hours.

The dosage form optionally comprises a third ensemble of pulsed-release pellets comprising a third average pulsed-release coating weight and having a third dissolution profile of a third T50, wherein the third T50 is greater than the first T50 and less than the second T50, that is, the third T50 is intermediate to the first and second T50s. Further, the dosage form optionally comprises fourth, fifth, sixth, seventh and eighth ensembles of pulsed-release pellets comprising a fourth, fifth, sixth, seventh and eighth average pulsed-release coating weight and having a fourth, fifth, sixth, seventh and eighth dissolution profile with a fourth, fifth, sixth, seventh and eighth T50, wherein the fourth, fifth, sixth, seventh and eighth T50 is different from the first, second and third T50.

In one embodiment, the pulsed-release pellets comprise a core having disposed thereon a core composition layer comprising an active agent and optionally a binder. In one embodiment, the core composition layer is disposed directly on the surface of the core. Exemplary cores include inert spheroids, Nonpareils, sugar spheroids, Cellets®, Celphere®, microcrystalline cellulose spheres, spheres made of microcrystalline cellulose and one or more sugars, such as lactose, and combinations comprising one or more of the foregoing cores. In one embodiment, the core is a sugar sphere. The average size of cores is, for example, about 250 μm to about 1500 μm. Commercially available sugar spheres are in US standard sieve size ranges of 14-16, 16-18, 18-20, 20-25, 25-30, 30-35, 40-60, for example. The cores comprise about 10 wt % to about 98 wt %, specifically about 20 wt % to about 90 wt %, and more specifically about 30 wt % to about 85 wt %, of the total weight of the core and the core composition layer.

In one embodiment, the pellets are formed by coating (e.g., spraying) the sugar spheres with an aqueous or non-aqueous solution or suspension that comprises the active agent. The active agent is coated onto the sugar spheres in the presence of, for example, a binder, a filler, a solubilizer, and other additives, and combinations comprising one or more of the foregoing additives. Suitable binders include, for example, polyethylene oxide, hydroxypropylcellulose, hydroxypropylmethylcellulose, ethylcellulose, cellulose acetate butyrate, hydroxypropylmethylcellulose phthalate, polyvinyl acetate phthalate, acacia, carboxymethylcellulose sodium, dextrin, gelatin, glucose, guar gum, hydroxyethyl, methylcellulose, polymethacrylates, povidone, pregelatinized starch, sodium alginate, zein, and the like, and combinations comprising one or more of the foregoing binders. In one embodiment, the binder comprises, a hydroxypropylcellulose, such as hydroxypropylcellulose NF 75-150 cps. Suitable suspension media comprise, for example, a solvent such as isopropyl alcohol, ethanol, water, and the like, and combinations comprising one or more of the foregoing solvents. The optional binder, when present, comprises about 0.1 wt % to about 20 wt %, specifically about 0.2 wt % to about 10 wt %, and more specifically about 3 wt % to about 8 wt %, of the total weight of the core and the core composition layer.

In one embodiment, the core is substantially free of an organic acid, i.e., the amount of such organic acid, if any, is sufficiently small so as not to substantially affect the release rate of the active agent from the core. Organic acids include, for example, adipic acid, ascorbic acid, citric acid, fumaric acid, malic acid, succinic acid, and tartaric acid.

The core composition layer comprises an active agent such as diltiazem, verapamil, propranolol, fluoxetine, venlafaxine, methylphenidate, amphetamines, zolpidem, and galantamine suitably in the form of a pharmaceutically acceptable salt. In one embodiment, the active agent is a water-soluble active agent. In some embodiments, the active agent is soluble in the dispersing agent employed in a rotary granulation process. Other suitable active agents include, anti-inflammatory substances, coronary vasodilators, cerebral vasodilators, peripheral vasodilators, anti-infectives, psychotropics, antimanics, stimulants, anti-histamines, gastrointestinal sedatives, anti-diarrheal preparations, anti-anginal drugs, vasodilators, antiarrythmics, anti-hypertensive drugs, vasoconstrictors drugs useful to treat migraines, anticoagulants and antithrombotic drugs, analgesics, anti-pyretics, hypnotics, sedatives, anti-emetics, anti-nauseants, anticonvulsants, neuromuscular drugs, hyper- and hypoglycaemic agents, thyroid and antithyroid preparations, diuretics, antipasmodics, uterine relaxants, mineral and nutritional additives, antiobesity drugs, anabolic drugs, erythropoietic drugs, antiasthmatics, expectorants, cough suppressants, mucolytics, antiuricemic drugs and other drugs.

Additional suitable active agents include gastrointestinal sedatives such as metoclopramide and propantheline bromide; antacids such as cimetidine; anti-inflammatory drugs such as phenylbutazone, indomethacin, naproxen, ibuprofen, flurbiprofen, diclofenac, dexamethasone, predinisone and prednisolone; coronary vasodilator drugs such as glyceryl trinitrate, isosorbide dinitrate and pentaerythritil tetranitrate; peripheral and cerebral vasodilators such as soloctidilum, vincamine, naftidorofuryl oxalate, co-dergocrine mesylate, cyclandelate, papaverine and nicotinic acid; anti-infective substances such as erythromycin stearate, cephalexin, nalidixic acid, tetracycline hydrochloride, ampicillin, flucloxacillin sodium, hexamine mandelate and hexamine hippurate; neuroleptic drugs such as flurazepam, diazepam, temazepam, amitryptyline, doxepin, lithium carbonate, lithium sulfate, chlorpromazine, thioridazine, trifluperazine, fluphenazine, piperothiazine, haloperidol, maprotilline hydrochloride, imipramine and desmethylimipramine; central nervous stimulants such as methylphenidate, ephedrine, epinephrine, isoproterenol, amphetamine sulfate and amphetamine hydrochloride; antihistamic drugs such as diphenhydramine, diphenylpyraline, chlorpheniramine and brompheniramine; drugs affecting the rhythm of the heart, such as verapamil, nifedipine, diltiazem, procainamide, disopyramide, bretylium toxylate, quinidine sulfate and quinidine gluconate; drugs used in the treatment of hypertension such as propranolol hydrochloride, guanethidine mono-sulphate, methyldopa, oxprenolol hydrochloride, captopril and hydralazine; drugs used in the treatment of migraine such as ergotamine; drugs affecting coagulability of blood such as epsilon aminocaproic acid and protamine sulfate; analgesic drugs such as acetylsalicyclic acid, acetaminophen, codeine phosphate, codeine sulfate, oxydodone, dihydrocodeine tartrate, oxycodeinone, morphine, heroin, nalbuphine, butorphanol tartrate, pentazocine hydrochloride, cyclazacine, pethidine, buprenorphine, scopolamine and mefanamic acid; anti-epileptic drugs such as phenyloin sodium and sodium valproate; neuromuscular drugs such as dantrolene sodium; substances used in the treatment of diabetes such as tolbutamide, disbenase glucagon and insulin; drugs used in the treatment of thyroid gland disfunction such as triodothyronine, thyroxine and propylthiouracil; diuretic drugs such as furosemide, chlorthalidone, hydrochlorthiazide, spironolactone and trimterene; the uterine relaxant drug ritodrine; appetite suppressants such as fenfluramine hydrochloride, phentermine and diethylproprion hydrochloride; antiasthmatic and bronchodilator drugs such as aminophyline, theophyline, salbutamol, orciprenaline sulphate and terbutaline sulphate; expectorant drugs such as fuaiphenesin; cough suppressants such as dextromethorphan and noscapine; mucolytic drugs such as carbocisteine; adecongestant drugs such as phenylpropanolamine and pseudoephedrine; hypnotic drugs such as dichloralphenazone and nitrazepam; anti-nauseant drugs such as promethazine theoclate; haemopoietic drugs such as ferrous sulphate, folic acid and calcium gluconate; uricosuric drugs such as sulphinpyrazone, allopurinol and probenecid; drugs useful for treating Crohn's disease, e.g., 5-amino salicyclic acid, and the like.

In one embodiment, the active agent is present in the dosage form as a pharmaceutical salt. “Pharmaceutically acceptable salts” includes derivatives of the active agent, wherein the active agent is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and combinations comprising one or more of the foregoing salts.

The active agent comprises about 20 wt % to about 90 wt %, more specifically about 30 wt % to about 85 wt %, of the total weight of the core and the core composition layer.

In one embodiment, the active agent-binder coating mixture is deposited on the core using a rotary granulation process. In this embodiment, the active agent-binder coating mixture is atomized onto a fluidized bed of cores located in the rotor granulator. Because of the difference in size between the cores and the atomized active agent-binder mixture, the active agent sticks to the cores and the binder retains the active agent on the cores. In the fluidized bed, a rotor-disk granulator makes the cores move with fluid-like motion. As the cores move within the fluidized bed, they are sprayed with the active agent-binder mixture until the desired quantity of active agent is deposited upon the cores. Coating of the active agent and binder is optionally followed by a drying step.

The core comprising the active agent is then coated with a pulsed-release coating composition. In one embodiment, the pulsed-release coating composition comprises a mixture of a relatively large proportion of a lubricant and a relatively small proportion of a wetting agent in admixture with a minor proportion of a first polymer that is permeable to the active agent and water and a major proportion of a polymer that is less permeable to the active agent and water than the first polymer. No organic acids, such as adipic acid, ascorbic acid, citric acid, fumaric acid, malic acid, succinic acid, tartaric acid and fumaric acid, are required to be included into the coating layer. A suitable permeable first polymer is the cationic polymer synthesized from acrylic and methacrylic acid ester with a low content of quaternary ammonium groups, known as EUDRAGIT® RL manufactured by Degussa. EUDRAGIT® RL is a copolymer of acrylic and methacrylic esters comprising quaternary ammonium groups and having a ratio of quaternary ammonium groups to neutral meth(acrylic) esters of 1:20. In this compound, the ammonium groups give rise to the permeability of the polymer. The permeability of EUDRAGIT® RL is reportedly independent of pH. A suitable less permeable second polymer is another such cationic polymer known as EUDRAGIT® RS manufactured by Degussa. EUDRAGIT® RS is less permeable than EUDRAGIT® RL because EUDRAGIT® RS has fewer ammonium groups. Eudragit RS is a copolymer of acrylic and methacrylic esters comprising quaternary ammonium groups and having a ratio of quaternary ammonium groups to neutral meth(acrylic) esters of 1:40. The permeability of EUDRAGIT® RS is reportedly independent of pH.

The pulsed-release coating layer alternatively comprises a mixture of polymers, synthetic and/or naturally occurring, that are freely permeable, slightly permeable, water soluble, water insoluble, and polymers whose permeability and/or solubility is affected by pH. In addition to those referred to above, such suitable polymers for inclusion into the coating layer include EUDRAGIT® S, EUDRAGIT® L, EUDRAGIT® E, polyvinyl alcohol, polyvinylpyrrolidone, and combinations comprising one or more of the foregoing polymers. Commercially available polymeric solutions and/or suspensions may also be employed. These solutions/suspensions may optionally contain plasticizing agents to improve the polymer characteristics of the coating. Examples of such solutions and/or suspensions include EUDRAGIT® RS30D, EUDRAGIT® RL 30D, EUDRAGIT® L 30D, EUDRAGIT® E 12.5, EUDRAGIT® RL 12.5 P, EUDRAGIT® RS 12.5, AQUACOAT® made by FMC Corporation, SURELEASE® made by Colorcon Inc., and combinations comprising one or more of the foregoing. AQUACOAT® is an aqueous polymeric dispersion of ethylcellulose and contains sodium lauryl sulfate and cetyl alcohol. SURELEASE® is an aqueous polymeric dispersion of ethylcellulose and contains dibutyl sebacate, oleic acid, ammoniated water and fumed silica.

In addition to the polymers, the pulsed-release coating layer includes a lubricant and optionally a wetting agent. Suitable lubricants include talc, calcium stearate, colloidal silicon dioxide, glycerin, magnesium stearate, mineral oil, polyethylene glycol, and zinc stearate, aluminum stearate, glyceryl monostearate, cetostearyl alcohol, cetyl alcohol, lanolin alcohols, stearyl alcohol, lecithin, mineral oil, and combinations comprising one or more of the foregoing lubricants.

Suitable wetting agents include sodium lauryl sulfate, acacia, benzalkonium chloride, cetomacrogol emulsifying wax, diethanolamine, docusate sodium, sodium stearate, emulsifying wax, hydroxypropyl cellulose, monoethanolamine, poloxamer, polyoxyethylene alkyl ethers, polyoxyethylene castor oil derivatives, polyoxyethylene sorbitan fatty acid esters, polyoxyethylene stearates, propylene glycol alginate, sorbitan esters and triethanolamine, and combinations comprising one or more of the foregoing wetting agents.

In one embodiment, the pulsed-release coating layer of the pellet comprises 2 wt % to 7 wt % of the first polymer more permeable to the active agent, 53 wt % to 59 wt % of the second polymer less permeable to the active agent, 0 wt % to 8 wt % of the plasticizer, 0 wt % to 8 wt % of the wetting agent, and 31 wt % to 35 wt % of the lubricant, expressed as percentages of the total weight of the pulsed-release coating layer.

The pulsed-release coating composition optionally comprises additional fillers such as, for example, talc, kaolin, calcium sulfate, and combinations comprising one or more of the foregoing fillers. In one embodiment the optional additional filler comprises talc. The amount of additional filler is about 15 wt % to about 200 wt %, specifically about 30 wt % to about 100 wt % of the weight of the polymers in the pulsed-release coating composition.

The pulsed-release coating optionally comprises a water-soluble or water-insoluble plasticizer. Exemplary water-soluble plasticizers include triethyl citrate, triacetin, polyethylene glycol, propylene glycol, sorbitol, glycerin, and combinations comprising one or more of the foregoing plasticizers. Exemplary water-insoluble plasticizers include dibutyl sebacate, diethyl phthalate, dibutyl phthalate, tributyl citrate, acetyl tributyl citrate, castor oil, mineral oil, glyceryl monostearate, and combinations comprising one or more of the foregoing plasticizers. The plasticizer comprises about 0 wt % to about 30 wt %, specifically about 5 wt % to about 15 wt %, of the total weight of the pulsed-release coating composition.

The pulsed-release coating composition is applied to the cores using a coating technique used in the pharmaceutical industry, such as fluid bed coating. Once applied and dried, the polymer content of the pulsed-release coating comprises about 5 wt % to about 35 wt % of the total weight of the coated cores, or about 7 wt % to about 25 wt % of the total weight of the coated cores. In this context, coated core means the pellet comprising the API plus any additional coatings.

The pulsed-release coating is optionally dried before applying an optional second coating. A color imparting agent is optionally added to the pulsed-release coating composition or a rapidly dissolving seal coat containing color may be coated over the sustained-release coating layer provided that the seal coat is compatible with and does not affect the dissolution of the sustained-release coating layer. Exemplary film-forming agents include polyvinylpyrollidone, hydroxypropyl cellulose, hydroxypropyl methylcellulose, polyethylene oxide, polyethylene glycol, and combinations comprising one or more of the foregoing film-forming agents.

The multiparticulate dosage form of the active agent is optionally encapsulated in hard gelatin to provide a desired quantity of active agent in an oral dosage form. Alternatively, the multi-particulate dosage form is formed into tablets, for example, by first adding about 40 wt % to about 90 wt % of a solid pharmaceutically acceptable tablet excipient which will form a compressible mixture with the coated cores and which may be formed into a tablet without crushing the coated cores, and optionally an effective amount of a tablet disintegrating agent and a lubricant. The solid pharmaceutically acceptable tablet excipient comprises, for example, carnuba wax, lactose, dextrose, mannitol, microcrystalline cellulose, kaolin, powdered sucrose, vegetable starches and combinations comprising one or more of the foregoing excipients. Suitable tablet disintegrants comprise, for example, crospovidone, croscarmellose sodium, dry starch, sodium starch glycolate, and the like, and combinations comprising one or more of the foregoing disintegrants. Suitable lubricants include, for example, calcium stearate, glycerol behenate, magnesium stearate, mineral oil, polyethylene glycol, sodium stearyl fumarate, stearic acid, talc, hydrogenated vegetable oil (e.g., Lubritab®), zinc stearate, and combinations comprising one or more of the foregoing lubricants.

Also included are methods of increasing patient compliance by administering dosage forms made by the disclosed method.

### EXAMPLES

The following examples further illustrate the invention but, of course, should not be construed as in any way limiting its scope. In particular, the processing conditions are merely exemplary and can be readily varied by one of ordinary skill in the art.

### Example 1

This example illustrates the release profiles obtained by coating diltiazem pellets with different coating weights. Pellets can be prepared in accordance with U.S. Pat. No. 5,834,024, incorporated herein by reference Diltiazem hydrochloride (e.g., 10.50 kg) and hydroxypropylcellulose (e.g., 6.25 kg) are mixed with ethanol (e.g., 16.250 L) to form a homogeneous slurry. Sugar spheres (e.g., 0.50 to 0.60 mm in diameter) (e.g., 5,000 kg) are added to a roto granulator and the slurry is applied. After about 2 hours, the active cores are recovered and dried for 30 minutes at 50° C. To form the coating solution, EUDRAGIT® RS (e.g., 3.1 kg) and EUDRAGIT® RL (e.g., 0.22 kg), triethyl citrate (e.g., 0.33 kg) and sodium lauryl sulfate (e.g., 0.075 kg) are dissolved in ethanol (e.g., 24.4 L). Talc (e.g., 1.86 kg) is added to the solution.

A single coating layer is applied to the active cores using a WURSTER bottom spray coater until the desired coat weight is achieved. The coated cores are then dried for 30 minutes at 50° C., cooled to room temperature, sieved through a 1400 μm sieve, and dusted with talc.

Dissolution profiles are measured in a USP Apparatus-2, pH 6.8 (phosphate buffer), 900 ml, 100 rpm, 37° C. FIG. 1 shows the dissolution profiles for ensembles of pellets having different coating weights, 7, 9, 11, 13, 15, 17 and 19 wt %. Blending a plurality of pellet ensembles with different dissolution profiles broadens the distribution of individuals, altering the rate of release.

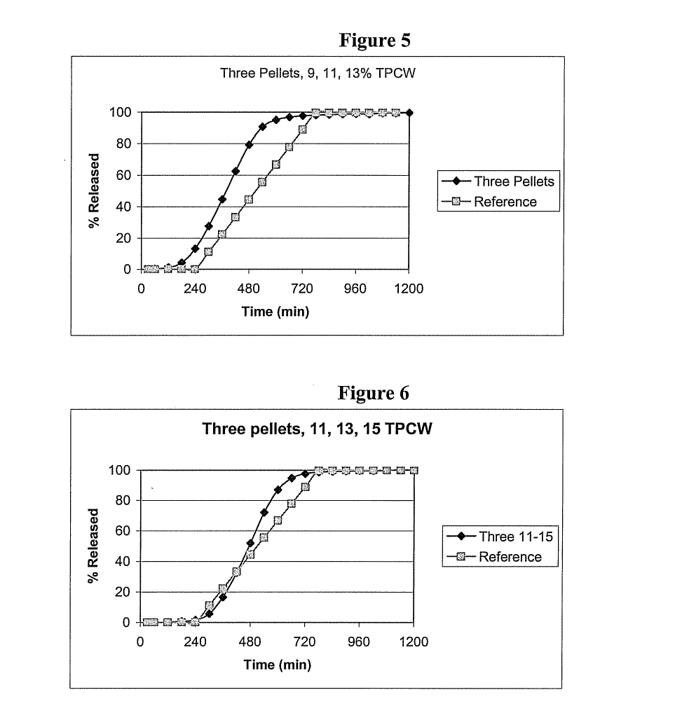

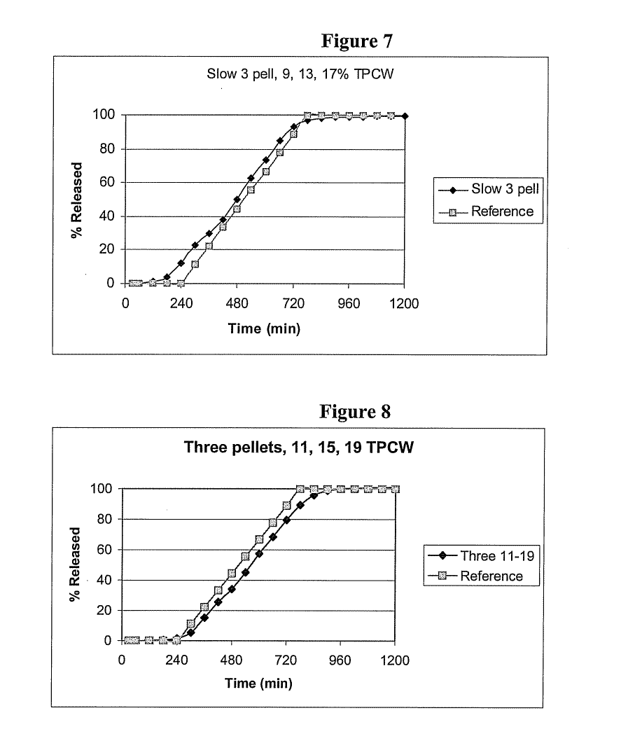

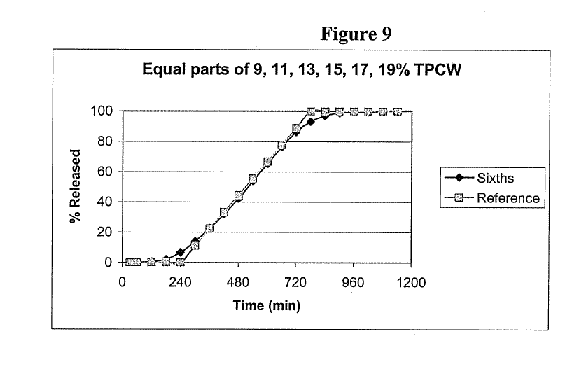

Blends of the diltiazem pellets result in an average profile of variable slope and lag time, depending which pellets are chosen. For demonstration, equal portions of pellets are shown in FIGS. 4-9. A reference profile having a lag of 180 minutes and 100% released in 720 minutes with perfect zero order release is included for comparison. The ability to vary the lag and the slope by various combinations of pellets gives the ability to meet any zero order release product, such as an osmotic pump. For example, after approximately 1-hour lag time the blend in FIG. 4 releases approximately 7.7% per hour for 11 hours, while the blend in FIG. 5 releases approximately 20% per hour for 4 hours after a 3-hour lag.

Disclosed herein are multiparticulate dosage forms having substantially zero-order release. An advantage of the disclosed dosage forms is that zero order release can be achieved in a multiparticulate dosage form. Advantages of multiparticulate dosage forms over single tablet dosage forms such as osmotic pump dosage forms include predictable gastrointestinal transit, particularly emptying from the stomach. In a multiparticulate dosage form, spreading out of the particles over the GI tract reduces irritation to the gut wall, and reduces the risk of “dose dumping”. In addition, as compared to osmotic pump dosage forms, there is not need for an overage in the amount of active agent in the dosage form to account for drug exclusion from the amount released. Also, the multiparticulate dosage form does not require the use of harsh solvents such as acetone or isopropyl alcohol required to form osmotic pump dosage forms. Thus, the multiparticulate dosage forms disclosed herein provide an advantageous equivalent to zero-order release osmotic pump dosage forms.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The term wt % refers to percent by weight. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of providing a zero-order release dosage form for an active agent, comprising:
combining n amounts of n ensembles of pulsed-release pellets, each of the n ensembles having a n^{th} dissolution profile with an n^{th} T50, to produce a combination ensemble of pellets that releases the active agent at a substantially constant rate following a lag time, wherein n is 2 to 8;
wherein the n^{th} ensemble of pellets comprises an n^{th} core having disposed thereon an n^{th} core composition layer, the n^{th} core composition layer comprising the active agent, and a n^{th} pulsed-release coating disposed on the n^{th} core composition layer;
wherein the lag time of release from the combination ensemble is 30 minutes to 8 hours; and
wherein at least two of the n ensembles of pulsed-release pellets have n^{th} T50s that differ by at least 4 hours.

2. The method of claim 1, wherein n is 3 to 8.

3. The method of claim 1, wherein n is 3 to 6.

4. The method of claim 1, wherein the first ensemble of pulsed-release pellets comprises a first average coating weight, wherein the second ensemble of pulsed-release pellets comprises a second average coating weight, and wherein the first average coating weight and the second average coating weight differ by 1 wt % or greater based on the weight of coating material.

5. The method of claim 1, wherein the n^{th} coating weight of each of the n ensembles of pulsed-release pellets differs from the other n−1 ensembles by 1 wt % or greater, wherein coating weights are based on the total weight of the coated pellets in each ensemble.

6. The method of claim 1, wherein the n^{th} pulsed-release coatings comprise 31% to 35% lubricant, 2% to 7% of a first copolymer of acrylic and methacrylic acid esters, and 53% to 59% of a second copolymer of acrylic and methacrylic acid esters, expressed as percentages of the total weight of the pulsed-release coating layer, the first copolymer being permeable to water and the active agent, the second copolymer being less permeable to water and the active agent than the first copolymer.

7. The method of claim 1, wherein the active agent comprises diltiazem, verapamil, propranolol, fluoxetine, venalfaxine, methylphenidate, zolpidem, or galantamine.

8. A method of increasing patient compliance, comprising providing a zero-order active agent dosage form to a human patient in need thereof, wherein the dosage form is produced by a method comprising:
combining n amounts of n ensembles of pulsed-release pellets, each of the n ensembles having a n^{th} dissolution profile with an n^{th} T50, to produce a combination ensemble of pellets that releases the active agent at a substantially constant rate following a lag time, wherein n is 2 to 8;
wherein the n^{th} ensemble of pellets comprises an n^{th} core having disposed thereon an n^{th} core composition layer, the n^{th} core composition layer comprising the active agent, and a n^{th} pulsed-release coating disposed on the n^{th} core composition layer;
wherein the lag time of release from the combination ensemble is 30 minutes to 8 hours; and
wherein at least two of the n ensembles of pulsed-release pellets have h T50s that differ by at least 4 hours.

9. The method of claim 8, wherein n is 3 to 8.

10. The method of claim 9, wherein n is 3 to 6.

11. The method of claim 8, wherein the first ensemble of pulsed-release pellets comprises a first average coating weight, wherein the second ensemble of pulsed-release pellets comprises a second average coating weight, wherein the first average coating weight and the second average coating weight differ by 1 wt % or greater, and wherein coating weights are based on the total weight of the coated pellets in each ensemble.

12. The method of claim 8, wherein the n^{th} coating weight of each of the n ensembles of pulsed-release pellets differs from the other n−1 ensembles by 1 wt % or greater, wherein coating weights are based on the total weight of the coated pellets in each ensemble.

13. The method of claim 8, wherein the first and second pulsed-release coatings comprise 31% to 35% lubricant, 2% to 7% of a first copolymer of acrylic and methacrylic acid esters, and 53% to 59% of a second copolymer of acrylic and methacrylic acid esters, expressed as percentages of the total weight of the pulsed-release coating layer, the first copolymer being permeable to water and the active agent, the second copolymer being less permeable to water and the active agent than the first copolymer.

14. The method of claim 8, wherein the active agent comprises diltiazem, verapamil, propranolol, fluoxetine, venalfaxine, methylphenidate, zolpidem, or galantamine.

15. An active agent dosage form comprising,
a combination ensemble of pellets, the combination ensemble comprising n amounts of n ensembles of pulsed-release pellets, each of the n ensembles having a n^{th} dissolution profile with an n^{th} T50, wherein the combination ensemble releases the active agent at a substantially constant rate following a lag time, wherein n is 2 to 8;
wherein the n^{th} ensemble of pellets comprises an n^{th} core having disposed thereon an n^{th} core composition layer, the n^{th} core composition layer comprising the active agent, and a n^{th} pulsed-release coating disposed on the n^{th} core composition layer;
wherein the lag time of release from the combination ensemble is 30 minutes to 8 hours; and
wherein at least two of the n ensembles of pulsed-release pellets have n^{th} T50s that differ by at least 4 hours.

16. The dosage form of claim 15, wherein the n^{th} pulsed-release coatings comprise 31% to 35% lubricant, 2% to 7% of a first copolymer of acrylic and methacrylic acid esters, and 53% to 59% of a second copolymer of acrylic and methacrylic acid esters, expressed as percentages of the total weight of the pulsed-release coating layer, the first copolymer being permeable to water and the active pharmaceutical ingredient, the second copolymer being less permeable to water and the active pharmaceutical ingredient than the first copolymer.

17. The dosage form of claim 15, wherein the active agent comprises diltiazem, verapamil, propranolol, fluoxetine, venalfaxine, methylphenidate, zolpidem, or galantamine.
